(19) United States
(12) Patent Application Publication
(10) Pub. No.: US 2006/0015157 A1
(43) Pub. Date: Jan. 19, 2006

(54) Method and apparatus for particle radiation therapy and practice of particle medicine

(76) Inventor: Vong Vui Leong, Las Vegas, NV (US)

Correspondence Address: TRASK BRITT, P.O. BOX 2550, SALT LAKE CITY, UT, 84110, US

(21) Appl. No.: 10/890,935

(22) Filed: Jul. 14, 2004

(51) Int. Cl. A61N 5/06; A61F 2/00; A61N 5/00

(52) U.S. Cl. 607/88; 607/100; 600/1

(57) ABSTRACT

A particle medicine slide designed with a chamber for securing a therapeutic material which allows the passage of a radiation beam through the transparent walls of the chamber and through a therapeutic material secured therein. The therapeutic material may be a solid, liquid or a gas, derived from inorganic and organic sources. A method of a treating a patient with therapeutic particle radiation by creating a beam of therapeutic particle radiation, placing a therapeutic material in the beam path of a radiation source, and directing the resulting therapeutic particle radiation onto meridian channels, trigger points, and acupuncture points located on a patient'body is also disclosed.

### FIELD OF THE INVENTION

The invention relates to a new field of medicinal particle therapy. More particularly, the invention relates to an apparatus that places medicinal particles in the path of a radiation source to a method of guiding the resulting therapeutic particles onto certain points of the human body, or other meridian points or trigger points of the body relating to the practice of acupuncture or acupressure, for the purpose of treating diseases and ailments.

### BACKGROUND

There are certain locations of the body, along meridian channels which, when subjected to acupuncture or acupressure treatment, will have a therapeutic affect on corresponding body functions, such as muscles, organ systems, and the like. For example, one specific acupuncture point on the ear may influence throat action, while another may influence mouth function or stomach activity. A common technique for acupuncture therapy is for the practitioner to pierce the skin and stimulate the acupuncture point with a fine needle. Another technique is acupressure therapy, practiced by applying pressure to acupuncture points. Alternative methods for the stimulation of these points are known, including the use of bone needles, electronic needles, electric current, pressure, heat, sound waves, or light. For example, U.S. Pat. No. 5,250,068 shows an acupuncture needle body which contains an optical fiber for transmitting light from a light source directly into the acupuncture site. U.S. Pat. No. 5,843,074 discloses a therapeutic device which uses pulsed and colored light applied to the skin over acupuncture points with an optical fiber. U.S. Pat. No. 5,261,422 shows a process and device for treatment of disease employing electromagnetic oscillations (i.e. sound waves) applied to the patient. U.S. Pat. No. 5,385,150 discloses an acupuncture device that directs electric pulses at acupuncture points. U.S. Pat. No. 4,535,784 shows an apparatus for stimulating acupuncture points with light radiation. U.S. Pat. No. 5,086,770 discloses an apparatus for producing a therapeutic radiation beam. Finally, U.S. Pat. No. 4,232,678 shows a device for stimulating acupuncture points by using an infra-red laser diode.

It would be desirable to provide an apparatus and method for creating therapeutic particle radiation and for exposing individuals to therapeutic particle radiation. It would also be desirable to provide an apparatus and a method of treatment that removes toxins from foods and medicines, thus avoiding bad side effects, while allowing the essential particles to be delivered to the patient through meridian channels and acupuncture points. Additionally, it would be desirable to provide a non-invasive method to treat diseases and maladies that may alleviate or eliminate the need for needles, oral medicines, and injection of medications.

### SUMMARY OF THE INVENTION

Certain foods and medicines may be ingested to aid in healing diseases and improving the longevity of an individual. When foods and medicines are ingested, it is believed that the human body may only extract a small portion of the beneficial particles. Moreover, many foods and medicines contain toxic substances that may counteract any benefit they may normally provide. As such, a person eating a complete diet may still become sick and age prematurely. It is believed that a full dosage of the beneficial and necessary particles may allow a return to full strength and increase average life expectancy. It is believed that a complete dosage of the necessary nutritional and medicinal particles may be delivered by directly introducing those particles into the meridian channels and acupuncture points on the human body. While much has been learned about the benefits of acupuncture and nutritional therapies, no previous approach has integrated these therapies in the manner of this invention. The present invention is believed to overcome the nutritional challenges of insufficient nutritional particles in our diet, as well as overcome the problems of toxicity in some food and medicines. This new method of therapy, combining acupuncture medicine and nutrition particles, is called particle medicine therapy.

Particle medicine therapy is based on the belief that, as a radiation beam passes through a sample of a therapeutic material, such as a solution of ginger root, the therapeutic material emits particles which are then picked up and carried by the exiting radiation beam. A radiation beam is a beam of electromagnetic radiation which may be released from a number of sources including visible white light, monochromatic light, lasers, microwaves, infrared light, ultraviolet light, Gamma rays, X-rays, radio waves, and other frequencies of the electromagnetic spectrum. The source of the radiation beam may be selected by the particle medicine practitioner according to the desired therapeutic material and the malady or disease being treated. It is believed the particles may be of many types, some even visible to the naked eye, such as the yellow particles that are emitted from Golden Seal after passing a light beam through a thin slice of Golden Seal.

When the radiation beam begins carrying the emitted particles, it becomes therapeutic particle radiation. The resulting therapeutic particle radiation can then be applied as particle medicine therapy to selected points on a patient's body. The selected points may include those meridian channel points used in acupuncture treatment. The type and form of the therapeutic material and the source of the radiation beam used for the particle medicine therapy may be determined by the specific condition or disease to be treated and the trigger points of the patient's body available for treatment.

The present invention also includes a particle medicine slide used to secure the therapeutic material in the path of a radiation beam. The particle medicine slide has a slide frame which forms a slide pocket and defines a chamber which is used to hold a therapeutic material. The chamber is made of transparent walls to allow the passage of a radiation beam through the therapeutic material contained therein. The particle medicine slide may be configured to hold the therapeutic material in a liquid, a gaseous, or a solid state. The shape and size of the particle medicine slide and the chamber may be configured to accommodate the particular needs of the therapy, (i.e., the chamber may be elliptical, spherical, cubical, conical, rectangular, prismatic, cylindrical, or pyramidal). The slide may be configured to allow long term storage of the therapeutic material. Alternatively, the slide maybe configured to allow the health care provider to make a fresh solution of the therapeutic material, place the fresh solution in the slide chamber, and cause the remainder of the solution to be ingested by the patient, or applied topically to the patient. With this approach the health care professional may utilize the full benefits of a therapeutic material.

The radiation beam may be produced by a number of thermal and optical sources across the electromagnetic spectrum. For example, the beam source may be a non-coherent light source such as a quartz-halogen-type light bulb, a monochromatic light emitting diode (LED), or polarized light. Additionally, the radiation beam source may be a coherent light source, for example, light waves with the same wave length and same phase, such as a diode laser or low power laser. The therapeutic material maybe a solid, a liquid solution, or a gaseous sample of any number of organic and inorganic materials collected from nature or synthetically derived. Some illustrative therapeutic materials may include Ginger Root, Penicillium, Rhei Rhizoma, Aurantii Fructus Immaturus, Magnoliae Officinalis Cortex, Cannabis Seed, Apricot Seed, Paeoniae Radix, Sesame Seed, Ophiogogonis Tuber, Glycyrrhizae Radix, Goldenseal Root, and other similar medicinal and nutritional compounds or combinations thereof, whether naturally or synthetically derived. Other therapeutic materials may include prescription drugs and other types of western medicines. It will be appreciated that this list is illustrative only and other suitable materials may be used and are within the scope of this invention.

It is believed that particle medicine therapy may play an important part of a holistic therapeutic approach to treat a variety of health problems and conditions. Particle medicine therapy, when used concurrently with nutritional therapy, surgery, physical therapy, or prescription medicines, may greatly contribute to the overall well being and recovery of the affected patient. Moreover, particle medicine therapy may enable a patient to quicken both the psychological and the physical processes necessary for a successful therapeutic approach. Moreover, it will be appreciated that particle medicine therapy includes the benefit of providing a perception of increased efficacy of accompanying treatments because of the personal interaction with health care professionals.

By the way of example, Golden Seal root may be used in a method of particle medicine therapy to treat diarrhea or constipation. The Golden Seal therapeutic particle radiation may kill pathogens associated with these maladies. Patients treated with this method may begin to experience normal bowel movements within 4 to 6 hours after particle medicine therapy. Furthermore, patients may have their symptoms alleviated without side effects (such as abdominal cramping) associated with traditional treatments.

It is also believed that the physical nature of particle medicine therapy, i.e. the use of radiation beams, allows the therapeutic particle radiation to be disseminated through fiber optic cables, such as those used in a telecommunications networks. For example, fiber optic cables carrying the therapeutic particle radiation may be connected to fiber optic cables in a telecommunication network, enabling the therapeutic particle radiation to be delivered through the fiber optic telecommunications cables over long distances to individual patients.

This invention includes devices and methods for the purpose of providing therapeutic particle radiation. Specifically, the invention is directed to a particle medicine slide designed with a chamber for securing a therapeutic material which allows the passage of a radiation beam through the transparent walls of the chamber and through a therapeutic material secured therein. The therapeutic material may be a solid, liquid or a gas, derived from inorganic and organic sources. Moreover, the invention is directed towards a method of a treating a patient with therapeutic particle radiation that includes creating a beam of therapeutic particle radiation by placing a therapeutic material in the beam path of a radiation source and then directing the resulting therapeutic particle radiation onto the meridians and acupuncture points located on a patient's body.

### BRIEF DESCRIPTION OF THE FIGS.

While the specification concludes with claims particularlypointing out and distinctly claiming that which is regarded as the present invention, the advantages of this invention can be more readily ascertained from the following description of the invention when read in conjunction with the accompanying drawings in which:

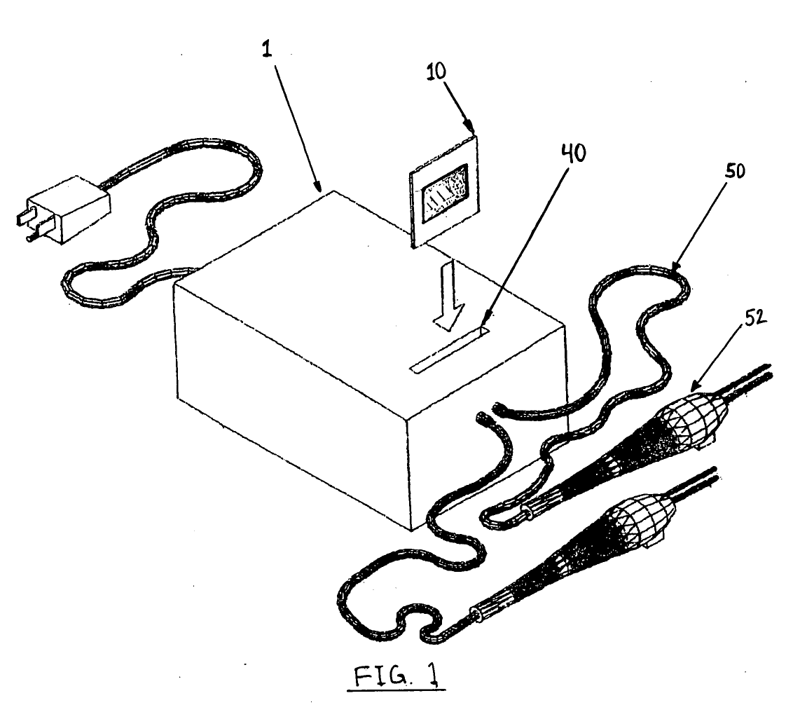

FIG. 1 is a drawing of an exemplary apparatus for creating therapeutic particle radiation demonstrating the placement of the particle medicine slide in the slide holder slot;

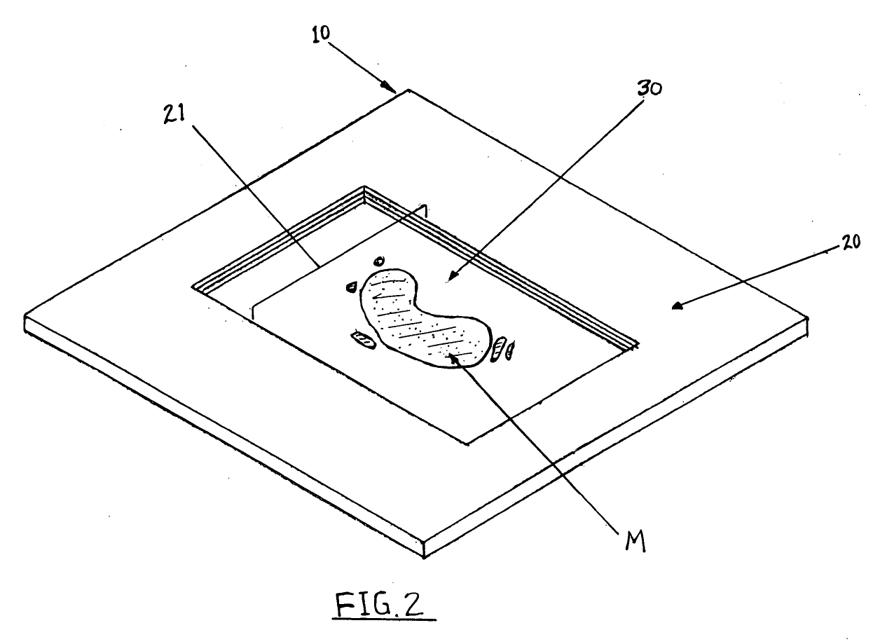

FIG. 2 is a view of one embodiment of a particle medicine slide with a slide frame defining a slide pocket, the slide pocket configured to define a chamber for containing a therapeutic material;

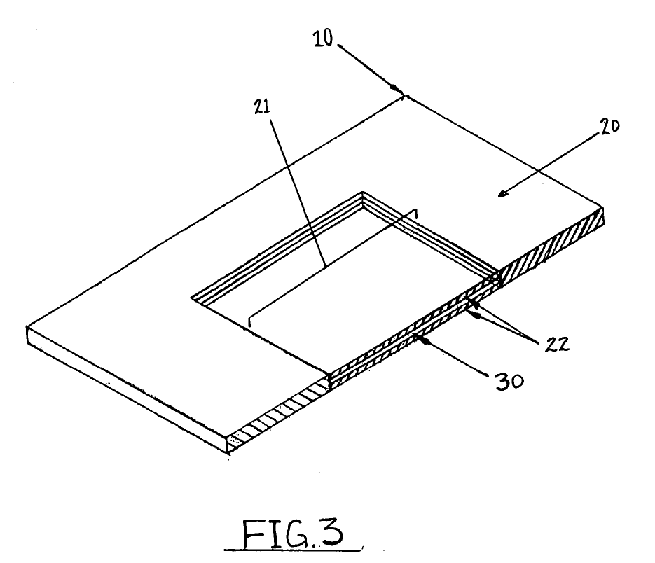

FIG. 3 is a cross sectional view of one embodiment of a particle medicine slide showing the slide frame and the transparent walls of the slide pocket, which define a chamber between the transparent walls; and

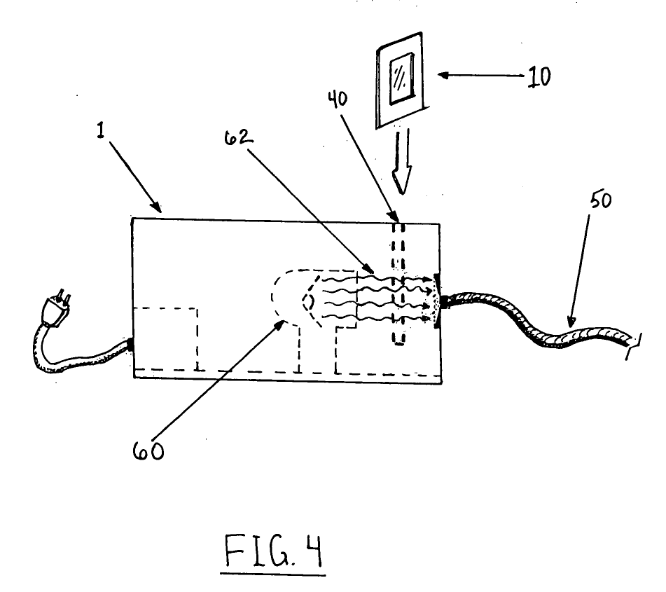

FIG. 4 is a cross sectional side view of an apparatus for creating therapeutic particle radiation demonstrating the placement of the particle medicine slide in the slide holder slot.

### DETAILED DESCRIPTION OF THE INVENTION

It will be appreciated that the following detailed embodiments described herein are illustrative only and do not limit the invention.

The method of particle medicine therapy includes the transfer of particles from therapeutic materials by means of a radiation beam onto the meridian channels and acupuncture points of the human body. Particle medicine therapy is believed effective due to a transfer of the necessary amounts of nutritional particles without a transfer of toxic or harmful particles from the therapeutic material. The transferred particles may be derived from a number of inorganic or organic therapeutic materials chosen for treatment by the doctor or healthcare professional. The radiation beam may be generated from any source along the electromagnetic radiation spectrum. The radiation beam may be a non-coherent light beam generated from a source such as a halogen light bulb, a coherent light beam generated from a source such as a laser, an infrared radiation beam generated by sources such as an infrared lamp, and other non-visible electromagnetic energy sources. The appropriate radiation beam source may be selected according to the ailment or disease for which the patient seeks treatment. For example, particle medicine therapy may be used to relieve maladies such as muscle numbness, paralysis, spasticity, tinnitus, deafness, rheumatoid arthritis, tendonitis, injury, asthma, diabetes, constipation, and premature aging. It will be appreciated that treatment with particle medicine therapy also includes the additional benefit of providing a perception of increased efficacy of other concurrent treatments because of the personal interaction with health care personnel.

Referring to the representative therapeutic particle radiation apparatus 1 and particle medicine slide 10 represented in FIGS. 1 through 4, a particle medicine slide 10 provides a means whereby a therapeutic material M can be contained and placed in the path of a radiation beam 62. The transparent walls 22 which define the chamber 30 allow the radiation beam to enter the chamber 30, pass through the therapeutic material M, and exit the chamber. As shown in FIGS. 1 and 4, the particle medicine slide 10 may be placed in a slide holder 40. This results in the slide chamber 30 and the therapeutic material M contained therein being placed in front of a radiation beam source 60 and the resulting radiation beam 62 generated by a radiation beam source 60. The passage of the radiation beam 62 through the therapeutic material M creates therapeutic particle radiation which exits the slide chamber 30 and are collected and directed into a fiber optic cable 50. The therapeutic particle radiation is then preferably directed along fiber optic cables 50 to end pieces 52, which can be used to direct the therapeutic particle medicine onto the meridian channels and acupuncture points on the body.

FIGS. 2 and 3 depict an illustrative embodiment of a particle medicine slide 10 with a slide frame 20, including an embodiment of a slide pocket 21 and the chamber 30. The slide frame 20 may be made of plastic, glass, wood, aluminum, stainless steel, or other formable material. The slide frame 20 may be substantially rectangular, oval, or trapezoidal. A slide pocket 21 is formed as an opening that passes through the slide 10, through the front and back planar surfaces.

A cross-sectional view of a slide 10 is shown in FIG. 3, depicting transparent pocket walls 22, which are separated from each other in the slide pocket 21. The transparent pocket walls 22, along with the perimeter of the slide pocket 21, form the boundaries of the chamber 30. The transparent pocket walls 22 may be constructed of plastic, glass, quartz crystal, or any other transparent material capable of forming the transparent walls. Chamber 30 may be configured to contain a therapeutic material M such as a fluid, a liquid solution, a solid, a gas, plasma, or a combination thereof. For example, chamber 30 may be sealed in such a manner to contain a liquid solution, a gas, or a solid. Chamber 30 may be configured in size and shape to accommodate differing sample sizes as desired.

Particular embodiments of a particle medicine slide 10 may allow placement of the therapeutic material M into the chamber 30 immediately prior to treatment, allowing treatment with fresh therapeutic material M. Suitable embodiments include a removable and re-sealable transparent pocket wall 22, a hinged first transparent pocket wall 22, an interchangeable chamber cartridge system, or a combination thereof. Another embodiment of the particle medicine slide 10 may include a slide frame 20 configured to be separable or to hinge open and closed from one side, effectively separating the first and second faces of the slide frame 20, to allow the insertion of a sealed chamber 30 in between the faces of the slide frame 20 and into the slide pocket 21. This embodiment would allow a number of sealed chambers 30 to be used interchangeably with a single hinged slide frame 20.

While the basis of the healing effect of the therapeutic particle radiation is not fully understood or explained by a modern theory, it is thought that many illnesses or maladies are attributed to an imbalance at the cellular level. The physiology of the affected cells is believed to be out of natural harmonious operation. It is postulated that medicinal particles emitted from the sample M are carried by the therapeutic particle radiation through the fiber optic cables 50 and directed to the meridian channels of the body through which the particles are delivered to the organs of the body where they interact with the malfunctioning cells, causing them to regain their natural balance. Moreover, the application of particle medicine therapy to acupuncture points may restore the balance and flow of energy through the meridian channels of the entire body, similar to the effects of traditional acupuncture therapy.

Although the present invention has been shown and described with respect to preferred embodiments, various additions, deletions and modifications that are obvious to a person skilled in the art to which the invention pertains, even if not shown or specifically described herein, are deemed to lie within the scope of the invention as encompassed by the following claims.

What is claimed is:

1. A particle medicine slide for securing a therapeutic material comprising:
a slide frame having a planar front face and a planar back face, the front face and the back face substantially parallel to each other;
a pocket defined by the inside perimeter of the slide frame; and
a chamber to secure a therapeutic material, the chamber defined by at least two transparent walls and the perimeter of the pocket.

2. The particle medicine slide of claim 1, wherein the chamber comprises:
a first transparent wall having a substantially planar front face and a substantially planar back face and a second transparent wall having a substantially planar front face and a substantially planar back face, the first and second transparent walls substantially parallel to one another; and
a separation between the first and second transparent walls configured to allow the holding of a therapeutic material such that a radiation beam may pass through the transparent walls of the chamber and through the therapeutic material.

3. The apparatus of claim 1, wherein the chamber comprises a means to allow the therapeutic material to be placed in the chamber

4. The apparatus of claim 3, wherein the means to allow the therapeutic material to be placed in the chamber comprises a removable first transparent wall or a hinged first transparent wall or a chamber cartridge system or a combination thereof.

5. The apparatus of claim 1 wherein the perimeter of the slide frame is substantially rectangular.

6. The apparatus of claim 1, wherein the transparent walls are plastic or glass.

7. A method of treating a patient with therapeutic particle radiation comprising:
placing a therapeutic material in a beam path of a radiation source;
passing radiation beams emitted by the radiation source through the therapeutic material to create therapeutic particle radiation; and
directing the resulting therapeutic particle radiation onto the patient's body.

8. The method according to claim 7, further comprising:
treating the patient with surgical, medicinal, or physical therapy while directing the resulting therapeutic particle radiation onto the patient's body.

9. The method according to claim 7, further comprising:
causing the patient to undergo intravenous administration, oral ingestion, topical application, sub-cutaneous administration, or rectal administration of the therapeutic material.

10. The method according to claim 7, wherein placing a therapeutic material in the beam path of a radiation source comprises placing the therapeutic material in the beam path of a non-coherent light source or a coherent light source.

11. The method according to claim 7, wherein directing the resulting therapeutic particle radiation onto the patient's body comprises directing the resulting therapeutic particle radiation onto meridian channels, trigger points, and acupuncture points located on a patient's body by means of a fiber optic cable.

12. The method according to claim 11, wherein directing the resulting therapeutic particle radiation onto meridian channels, trigger points, and acupuncture points located on a patient's body comprises use of an optical transmission fiber optic needle.

13. The method according to claim 7, wherein placing a therapeutic material in the beam path of a radiation source comprises placing a material selected from the group consisting of raw, diluted, liquid, gaseous, or solid ginger root, Penicillium, Rhei rhizome, Aurantii Fructus Immaturus, Magnoliae Officinalis Cortex, Cannabis Seed, Apricot Seed, Paeoniae Radix, Sesame Seed, Ophiogogonis Tuber, Glycyrrhizae Radix, Golden Seal Root and any combinations thereof.

14. A kit for exposing a patient to therapeutic particle radiation comprising:
a radiation source for generating a radiation beam, the radiation source comprising at least one of a thermal radiation source or optical radiation source;
a particle medicine slide comprising a pocket and a chamber with at least two transparent walls configured to hold a therapeutic material and allow the radiation beam to pass through the transparent walls and pass through the therapeutic material and allow the exit of therapeutic particle radiation;
a slide holder wherein the particle medicine slide is secured, the slide holder configured to allow the radiation beam to pass through the therapeutic material held inside the particle medicine slide chamber;
at least one fiber optic cable configured to direct the therapeutic particle radiation onto the patient's body; and a therapeutic material which may be held in the chamber and exposed to the radiation beam.

15. The kit claim of 14, wherein the therapeutic material is selected from the group consisting of orally ingestible therapeutic materials, intravenously administrable therapeutic materials, topically applicable therapeutic materials, sub-cutaneously administrable therapeutic material, rectally administrable therapeutic material and any combinations thereof.

16. The kit of claim 14, wherein the radiation beam source consists of a non-coherent light source or a coherent light source.

17. The kit claim of 14, wherein the therapeutic material is selected from the group consisting of Ginger Root, Penicillium, Rhei Rhizoma, Aurantii Fructus Immaturus, Magnoliae Officinalis, Cortex, Cannabis Seed, Apricot Seed, Paeoniae Radix, Sesame Seed, Ophiogogonis Tuber, Glycyrrhizae Radix, and Goldenseal Root and any combinations thereof.

18. The kit of claim 14, wherein the at least one fiber optic cable comprises an optical transmission fiber optic needle connected to the fiber optic cable.
